(19) United States
(12) Patent Application Publication
(10) Pub. No.: US 2021/0174304 A1
(43) Pub. Date: Jun. 10, 2021

(54) SYSTEM AND METHODS FOR COLLABORATIVE RESEARCH

(71) Applicant: ELIXSYS, INC., San Diego, CA (US)

(72) Inventor: Himabindu Gaddipati, San Diego, CA (US)

(21) Appl. No.: 16/768,699

(22) PCT Filed: Dec. 3, 2018
(86) PCT No.: PCT/US18/63694

§ 371 Date: Aug. 3, 2020

Related U.S. Application Data
(60) Provisional application No. 62/593,253, filed on Dec. 1, 2017.

(51) Int. Cl. G06Q 10/10; G06F 16/2453; H04L 29/06

(52) U.S. Cl. CPC G06Q 10/103; G16H 80/00; H04L 65/403; G06F 16/24539

(57) ABSTRACT

The present invention provides systems and computer-implemented methods for organizations to form collaborative research groups with other organizations, to share information, documents and data, to manage projects, and to participate in real-time collaborative activities.

### RELATED APPLICATIONS

This application claims the benefit of priority under 35 USC § 119 of U.S. Provisional Applications Ser. No. 62/593,253 filed Dec. 1, 2017, the entire disclosure of which is incorporated herein by reference.

### FIELD OF THE INVENTION

The present invention relates to a collaborative research and project management system, and in specific the invention relates to a method and system for different organizations or entities to interact, collaborate for different activities and exchange information on a platform for monitoring, accessing and automaticly capturing of information in relation to holistic research, development, products and services in different areas, such as oncology and the like.

### BACKGROUND OF THE INVENTION

Large-scale research and development, product manufacturing, financing and other aspects of bringing an idea to commercial fruition typically involves the participation of multiple stakeholders with complementary and competing capabilities. Stakeholder diversity in this process helps to keep price competitive and manages risk that a particular stakeholder will fail. While diversification undoubtedly yields short- to mid-term benefits, it fails to maximize long-term benefits by itself, because competing stakeholders traditionally work in silos with the intent of optimizing their own areas without having a holistic view of overall business needs.

Product development requires successful management of the complex interplay of technology, research, intellectual property, funding, regulation, manufacturing, commercialization and competitive intelligence. Each of these specialized areas must be monitored separately and cooperatively to successfully develop a product. At all the stages of development, diverse, current information is required to make appropriate product development decisions. Failure to discover and integrate this diverse information, or communicate it to relevant parties in a timely manner can lead to continuing development of flawed or unsound products and potentially the early termination of the viable ones.

Prior art research and development platforms do not exist with capabilities of engaging different organizations such as stakeholders or companies or groups to organize different research sectors and other sectors in a collaborative manner related to a particular domain. The organizations have no support for implementing the research sections in a sequential way without departing from their individual interests.

However, conventional platforms do not facilitate communication and information exchange among organizations. The platforms do not provide automatic updating and indexing of information that facilitates the organizations to monitor information specific to their needs. The platforms do not provide a single unifying portal to access the information and services pertaining to a specific domain, The platforms also do not facilitate organizations to form into groups.

Therefore, there is a need for a collaboration system for research to facilitate communication and information exchange among organizations or individuals. There is a need for an online portal that provides automatic updating and indexing of information to facilitate monitoring of information specific to needs in a user-friendly manner. There is a need for a single unifying platform to access the information and services pertaining to the oncology community or to other specific domains that involves any or all aspects of basic science research, product development, project management, manufacturing, commercialization, funding, regulation, IP and clinical care. Further, there is a need for a platform that facilitates formation of groupings of organizations and establishment of collaborations between the groups.

### SUMMARY OF THE INVENTION

The present invention provides a system for research collaboration including a) a non-transitory computer-readable storage medium storing a plurality of modules, each module comprising instructions that when executed on a processor (which can be cloud-based), cause the processor to perform a series of steps. wherein the plurality of modules comprises: a storage module, a dynamic display module, an entry module, an initiation module, a sharing module, an intelligent query module and a group activity module; and b) a database. The storage module is configured to receive, index, and store information on the non-transitory computer-readable storage medium and to retrieve indexed and stored information from the non-transitory computer-readable storage medium. In some aspects, the information is stored in the database, which can be a relational database. In certain aspects, the database can be linked to remote data storage on a cloud-based server.

The dynamic display means is configured to render a graphical user interface on a display device, which can be selected from the group consisting of a computer monitor, a tablet, a smart television and a mobile phone. The graphical user interface can be a map view, a bubble view or a group view.

The entry module is configured to receive categorical information about organizations, sub-organizations and individuals, and to authenticate and register the organizations, sub-organizations and individuals, thereby permitting access to the all or a portion of the modules of the system.

The initiation module is configured to initiate communication, projects, and programs between organizations, sub-organizations and individuals.

The sharing module is configured to enable organizations, sub-organizations and individuals to exchange information. The intelligent query module is configured to perform query-based searches and locate, store and retrieve information relevant to a query.

The intelligent query module perceives, learns, remembers and adapts queries based on query history and user input, and can use artificial intelligence.

The group activity module, which can include a create group module and a join group module, is configured to allow organizations, sub-organizations and individuals to create new groups and to join existing groups. The organizations, sub-organizations and individuals within a group can initiate and participate in real-time collaborative activity among other organizations, sub-organizations and individuals in the group, such as document sharing, data sharing, entering into collaborative projects, managing collaborative projects. messaging via message boards, direct messaging, audio conferencing and video conferencing.

Organizations, according to the systems of present invention, can be selected from colleges; universities; medical schools research institutes; foundations; human and veterinary hospitals; human and veterinary clinics; pharmaceutical companies; and biotechnology companies; medical device research, development and manufacturing companies; diagnostic laboratories; reference laboratories; contract research organizations; government institutes; government laboratories; government agencies; venture capitalist firms; investment banks; philanthropic groups; philanthropic societies; disease-focused patient groups; and professional associations.

Sub-organizations, according to the systems of the invention, can be selected from: a university department; a hospital department; a company department; a division; a subsidiary; a regional campus; a regional office; a regional building; regional satellite; an individual research laboratory; and a core shared facility operation.

Individuals of systems of the invention include professionals; physicians; surgeons; specialists; scientists; veterinarians; officers; directors; managers; stakeholders; administrators; consultants; attorneys; content contributors; curators; librarians; moderators; involuntary supporters; voluntary supporters; researchers; investigators; technicians; professors; lecturers; undergraduate students; graduate students; medical students; inventors; project managers; grant writers; and technical writers.

The present invention also provides computer-implemented methods for collaboration between organization, comprising the steps of a) registering two or more organizations for access to the system; b) creating a profile for each organization, wherein the profile includes information selected from the group consisting of: the name of the organization, the type of organization, the domain or domains of the organization, the address of the organization, the size of the organization, the qualifications, expertise and proficiencies of the organization, the research interests of the organization, the resources of the organization, and the needs of the organization; c) dynamically displaying all or a portion of each organization's profile in a map view, a bubble or cluster view, a list view or tabular view; and d) creating at least one group of the two or more organizations, wherein the organizations in the group initiate at least one real-time collaborative activity (e.g. document sharing, data sharing, entering into collaborative projects, managing collaborative projects, messaging via message boards, direct messaging, audio conferencing and video conferencing).

Organizations according to the invention can include sub-organizations and individuals, and can be, for example colleges; universities; medical schools; research institutes; foundations; human and veterinary hospitals; human and veterinary clinics; pharmaceutical companies; and biotechnology companies; medical device research, development and manufacturing companies; diagnostic laboratories; reference laboratories; contract research organizations; government institutes; government laboratories; government agencies; venture capitalist firms; investment banks; philanthropic groups; philanthropic societies; disease-focused patient groups; and professional associations.

Sub-organizations according to the methods of the invention can be, for example, a university department; a hospital department; a company department; a division; a subsidiary; a regional campus; a regional office; a regional building; regional satellite; an individual research laboratory; and a core shared facility operation.

Individuals according to the methods of the invention include professionals; physicians; surgeons; specialists; scientists; veterinarians; officers; directors; managers; stakeholders; administrators; consultants; attorneys; content contributors; curators; librarians; moderators; involuntary supporters; voluntary supporters; researchers; investigators; technicians; professors; lecturers; undergraduate students; graduate students; medical students; inventors; project managers; grant writers; and technical writers.

The present invention also provides systems for research collaboration comprising a computer-readable medium comprising instruction that when executed on a processor, perform the steps of: a) registering at least one organization on the system; b) storing information about the registered organization in a profile on a non-transitory computer-readable storage medium; and c) rendering and dynamically displaying all or part of the information in the profile. The display of all or part of the information can be a map view, a bubble or cluster view, a list view or tabular view of\

Registering at least one organization on the system can include the steps of: a) selecting an entry module comprising instructions that when executed on a processor, cause the processor to: i) prompting the organization to enter authenticating information selected from the group consisting of name, address, mail ID, a security code, and domain of interest; ii) comparing the authenticating information to authentic information for the organization; iii) authenticating the organization when the information of i) and ii) match; and iv) generating a user ID and password; b) entering additional profile information of the organization; c) storing the profile information, user ID and password to an indexed location on a non-transitory computer-readable storage medium.

The system can further include conducting a search by executing the steps of: a) entering a search query; b) initiating a search to retrieve information related to the search query; c) applying an automated learning algorithm to retrieved information to generate a focused search; d) retrieving and displaying the results of the focused search; and e) storing the focused search results to an indexed location on a non-transitory computer-readable storage medium.

The system can also create group by executing the steps of: a) selecting at organizations to form a group; b) adding the selected organizations to a queue; c) saving a profile of the group of selected to organizations to an indexed location on a non-transitory computer-readable storage medium; and d) beginning a real-time collaborative activity among the organizations in the group such as sharing documents, sharing data, entering into collaborative projects, managing collaborative projects, messaging via message boards, messaging directly, conferencing via audio and conferencing via video.

In other embodiments of the system organizations can join an existing group by carrying out the steps of: a) requesting to join the existing group; b) receiving an acceptance response from the existing group; c) adding the organization to the existing group; d) saving a modified profile of the group that includes the added organization to an indexed location on a non-transitory computer-readable storage medium; and e) initiating a real-time collaborative activity among the organizations in the group.

### BRIEF DESCRIPTION OF THE DRAWINGS

The accompanying drawings, which are incorporated in and constitute a part of the specification, illustrate embodiments of the invention, and together with the description, serve to explain the principles of the invention.

FIG. 1 is a block diagram of a collaboration system according to an embodiment of the invention with a specialized database and collaboration platform.

FIG. 2 is a flowchart for registering with a system according to an embodiment of the invention and creating a profile.

FIG. 3 is a flowchart for retrieving information from the system using an intelligent query module of the invention.

FIG. 4 is a flowchart of a process for joining a pre-existing group according to an embodiment of the invention.

FIG. 5 is a flowchart of a process for organizations and individuals to creating a new group.

### DETAILED DESCRIPTION

### Definitions

Unless otherwise noted, the terms used herein are to be understood according to conventional usage by those of ordinary skill in the relevant art.

It is to be understood that as used in the specification and in the claims, “a” or “an” can mean one or more, depending upon the context in which it is used. Thus, for example, reference to “a cell” can mean that at least one cell can be utilized.

“Module” as used herein, refers to any of a number of distinct but interrelated software units, that each contain everything necessary to execute an aspect of a desired functionality, from which a program may be built up. A module is a piece of an individual program, which can contain one or several routines.

As used herein, “organizations” are broadly defined to include without limitation, colleges; universities; medical schools; research institutes; foundations; hospitals and clinics (human and veterinary); pharmaceutical and biotechnology companies of all stages (e.g., discovery, development, translation, manufacturing and the like); medical device research, development and manufacturing companies; diagnostic and reference laboratories; contract research organizations; government institutes, laboratories and agencies; venture capitalist firms and investment banks; philanthropic groups and societies; disease-focused patient groups; professional associations and the like. The term organizations as used herein, can include and refer to suborganizations and individuals affiliated with the organization, and the terms are used interchangeably herein.

Exemplary “suborganizations” contemplated by the invention include departments (e.g. within a university, hospital, or company); divisions; subsidiaries; regional campuses, offices, buildings and satellites; individual research laboratories (e.g. within a university or institute); core shared facility operations (e.g., genomics, sequencing, microscopy, imaging, bioinformatics).

An “individual” include be any person employed, associated or affiliated with an organization or suborganization described herein, including but not limited to professionals (physicians, surgeons, specialists, scientists, veterinarians); officers and directors; managers; stakeholders; administrators; consultants; attorneys; content contributors; curators; librarians; moderators; supporters (involuntary and voluntary); researchers; investigators; technicians; professors; lecturers; students (e.g. undergraduate, graduate and medical students); inventors; project managers; grant and technical writers.

“Domain” as used herein, refers to an area of interest, expertise and resources. Domains are contemplated to include overlapping and interrelated areas of interest, expertise and resources, with an infinite number of levels of specificity. For example. the domain of oncology could include broadly, clinical focus and research focus subdomains. The clinical focus may include sub-subdomains of surgery, chemotherapy, radiation therapy, etc. that may also be organized in different ways such as by sub-subdomains of hematology, neurology, pulmonology, hepatology, etc. Each subdomain may overlap with non-oncology fields and interests as well. An infinite number of interrelated and overlapping subdomains are contemplated by the invention.

As used herein, terms “cluster”, “clustering” and “hierarchical clustering”, also known as “hierarchical cluster analysis”, refers to a process and algorithm that groups similar objects of information into groups called clusters. The endpoint is a set of clusters, where each cluster is distinct from each other cluster, and the objects within each cluster are broadly similar to each other. When rendered to a representational display, the proximity of one cluster to another thus reflects the relatedness of the information rather than geographic distances. Other parameters of the information clustered, such as relative magnitude and importance, can also be represented in cluster displays by such visual cues as color and size. In complex clustering, 2, 3 or more parameters can be analyzed to group and identify similarities and differences between information objects.

“Platform” as used herein, refers to the environment in which a piece of software is executed. It includes the operating systems and interfaces thereto, particularly the graphical user interface with which a user interacts to access the system of the invention, typically by remote access via the internet. Access can, in some aspects, include private network and virtual private network connections. Software executed according to the invention is organized into modules as described herein, which are instructions executed by a processor and memory to perform the basic functions of the system. Additional modules may be added to the system, and/or may access other non-system resources such as widgets, services, plug-ins and the like.

The present invention provides a collaboration system that allows different organizations to interact, collaborate on different activities and exchange information on a single unifying platform. The system includes a specialized database for storing data about and from organizations, sub-organizations, individuals and a collaboration platform through which the organizations, sub-organization and individuals can form intra- and inter-organizational groups to collaborate on and manage projects, and a platform that interacts with a processor and a memory configuration that executes certain instructions pertinent to information different organizations are interested in overall and for specific project. In certain embodiments, the processor and a memory are cloud-based. The system also includes components involved in executing instructions on a non-transitory computer-readable storage medium storing a plurality of modules, each module comprising instructions that when executed on a processor, cause the processor to perform a series of steps, where the plurality of modules comprises: a storage module, an entry module, an initiation module, a sharing module, an intelligent query module and a group activity module; and a dynamic display means that is adapted to render a graphical representation of information stored within the database to enable a plurality of organizations to communicate, display information in various formats and interact with the system.

The system is configured to present information to organizations based on their specific needs. Organizations related to a particular domain, such as oncology, are identified through a manual or automated search of public records e.g., on the internet, which can subsequently be updated and supplemented to include additional information (including confidential information) obtained from the organizations themselves and from other sources. Basic information about the organization is entered into the database, including the name of the organization, primary address, phone number, corporate status (e.g. C-Corp, S-Corp, Non-profit, etc.), “owner” (e.g., CEO, President, administrator or proprietor) name and title, type of organization, size parameters of the organization (e.g., number or employees, hospital beds, square feet and the like), expertise and interests. Sub-organizations (as defined above) are also entered and linked to the primary organization, as are individuals affiliated to the organization.

The organization and its sub-organizations can be represented in a variety of ways on a computer display. In Map View, the organization's location is shown geographically on a World Map. Zooming-out on the Map View displays the sub-organizations, such as international and satellite locations of the organization. Zooming-in on each location, accesses additional details about the sub-organization, which can be interactively accessed and displayed. Group View displays information in tabular information form, such as departments, research interests, individuals affiliated with the organization and the like. Bubble View displays organizations as clusters that are agnostic to geography across the world. Clusters represent an organization by selected parameters, such as size, expertise, interests with those organizations having the closest match to the selected parameter(s) prominently shown by size, color, and proximity within a cluster. Each view provides a distinct graphical user interface (GUI) that includes tools that permit searching, retrieval, sorting, filtering, and displaying organization-specific details and information. Multiple organizations can be grouped according to domain and the display dynamically updated.

Once entered into the system database, an organization is invited to join. For example, owners, administrators, CEOs and other individuals in the organization are identified and an invitation sent by email, postal mail or another method. Instructions are provided to log onto the system and access the entry module for registration of the organization through input an authentication of proper credentials, thereby create a profile for the organization. The individuals affiliated with the organization can enter additional contact, professional, personal and other information, including, e.g. links to data, publications, slide presentations and the like into their profile. Security is provided at various levels. For example, the organization owner may have access to certain information on all the profiles within the organization, while each individual profile can be set to restrict access to information to and from, within and outside of the organization.

Additional modules of the system provide access to a variety of functions. The initiation module is configured to initiate discussions, projects, and programs by and between different organizations and individuals. The sharing module is configured to enable organizations to exchange selected information with other organizations of the system, and between individuals and sub-organizations in the same organization. The intelligent query module is configured to perform query-based search through which relevant or target information is automatically retrieved, updated, indexed and displayed in a selected view with respect to the query entered. This module may employ artificial intelligence principles and algorithms designed to improve accuracy of future searches and to discover correlations automatically. The storage module is configured to store information uploaded by individuals, retrieved through searching, and used by the system. The group activity module is configured to allow organizations to join a pre-existing group or to create a new group of individuals. sub-organizations and organizations with shared collaborative interest.

In an aspect of the invention, the intelligent query module retrieves information related to an entered query, from various sources, and stores the information in the storage module and displays the information. The information can be, for example, related to a specific domain such as oncology. The information may be related to different research sections, product development, manufacturing, commercialization, funding, IP, project management, clinical care or combinations thereof. In an aspect of the invention, the intelligent query module applies an automated learning algorithm such as artificial intelligence methodologies or self-learning methodologies to identify the information most relevant to the entered search input, to display the information and to store it in the storage module.

In an aspect of the invention, the collaboration research platform allows organizations to access group-specific profiles through which information can be viewed, discussed and shared among organizations. The information available to other organizations can be limited by the profile owner. For example, the profile owner can specify information to be public, that is available to any other individual or organization in the system; semi-private, which is information accessible only to individuals and groups designated by the profile owner; or restricted and available only to the profile owner. Moreover, the invention contemplates assigning embargo dates upon which restricted information becomes semi-private or public, and expiration dates upon which semi-private and public information reverts to restricted access.

In another aspect of the invention, the collaboration research platform engages various organizations, sub-organizations, individuals, groups and the like to organize different research sectors in a collaborative manner related to a particular domain. The platform supports the stakeholders to implement the research sections in a sequential way without departing from their individual or organizational interests thereby resulting in the overall community growth.

The present invention will now be described in reference to the accompanying exemplary drawings. Wherever possible, same or similar reference numerals are used in the drawings and the description to refer the same or like parts or steps.

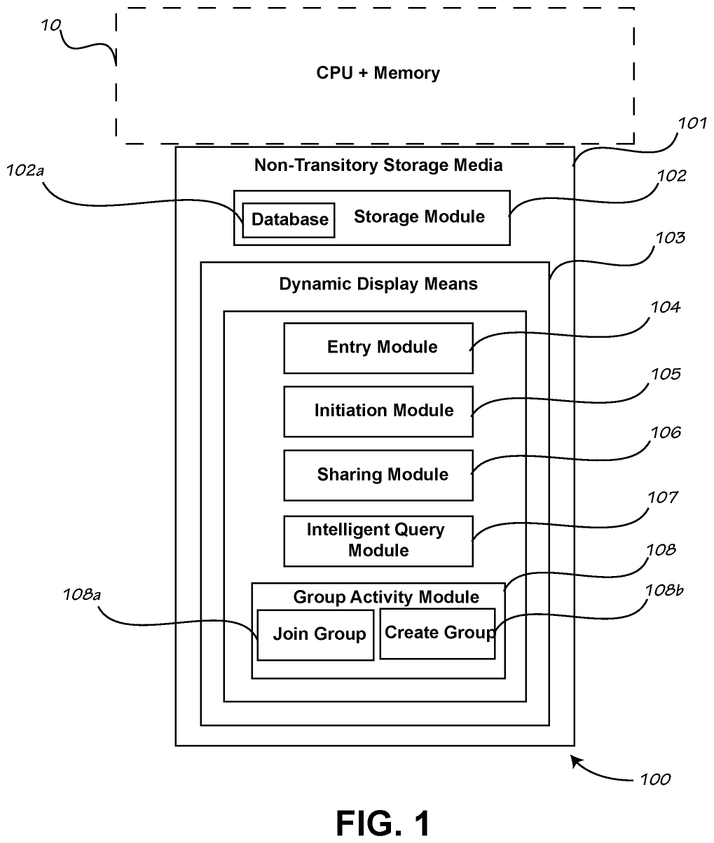

in an exemplary embodiment of the invention, the research collaboration system of the invention provides a comprehensive interactive platform, which operates through integrated modules. FIG. 1 is a block diagram of a collaboration system with a specialized database 102a and collaboration platform 100, comprising a non-transitory storage media 101 containing instructions stored therein, which is in communication with a processor and memory configured to execute the instructions. In certain aspects, the system can include the processor and memory 10. More typically, the processor and memory are provided by a server, which can be remotely located or in the “cloud” and are thereby external to system 100. The system includes storage module 102, which includes the database 102a to store information uploaded and retrieved through searching, and stores information used by the system and by components thereof. Additional storage space, if needed, can be provided by external databases (not shown) such as those on a cloud server, a google drive or the like, and which are interfaced to and in communication with the storage module 102. The dynamic display means 103 is configured to render a graphical user interface on remote display devices to enable the plurality of organizations to communicate, display information and to interact with the system. The dynamic display means 103 further comprises an entry module 104, an initiation module 105, a sharing module 106, an intelligent query module 107, and a group activity module 108.

The entry module 104 is configured to access the other modules of the system through registration of an organization by the input of proper credentials by the organization, thereby creating a profile, and for receiving generated means (i.e., user ID, password, security code, and other validation information) for entry into the profile. The initiation module 105 is configured to initiate collaborative discussions, projects, and programs by different organizations. The sharing module 106 is configured to be accessed by an organization to exchange information with other organizations of the system, and an intelligent query module 107 configured to perform query-based searches and to retrieve, store and transfer information relevant to the query entered by the organization.

The group activity module 108 is configured to authenticate organizations, which may then either join a pre-existing group using join group module 108a and/or create a new group using the create group module 108b. The join group module 108a comprises a collection of groups from which the organization can select to join. The create group module 108b comprises a collection of registered organizations which can be selected by an organization to form a group.

In accordance with the invention, the processor can be, for example, a general-purpose central processing unit (CPU), such as a microprocessor, or a special-purpose processor, such as a microcontroller. The memory can include, by way of examples but not by limiting random access memory (RAM), such as dynamic RAM (DRAM) and static RAM (SRAM). The memory can be local, remote, or distributed. In certain embodiments, the CPU and RAM are located on a remote computer that is interfaced to and in communication with the system. In other embodiments, the system includes the processor and/or RAM, such as an owned and operated local server that communicates through the internee or by another means of connecting to organizations remotely.

In accordance with one aspect of the invention, the system 100 facilitates organizations, suborganizations, individuals, and the like to interact with one another, to engage, collaborate, view and share information, documents, reports and the like and also retrieves information using the intelligent query module 108 of the system.

As illustrated, in FIG. 1, the dynamic display means 103 of the system is configured to combine the display of different sectors associated with the platform such as research, investments, clinical trials, components used for trials and the like in a single graphical image. The dynamic display means 103 is configured to simultaneously apply multiple filters. The dynamic display means 103 may render information as a raw data, analyzed data, organized data or as a figure, graph or any combination thereof. The dynamic display means 103 representations may be varied for a specific organization, and may be customized to specific organization's preferences or to particular needs. The dynamic display means 103 can further include features such as simplifying data, thereby representing potentially huge amounts of relevant data in a simpler manner, which can be more attractive, easier to view and more intuitive to use. The display can be interactive, thereby allowing the user to select the amount of simplification or complexity viewed.

The entry module 104 of the system 100 is the means by which organizations register with the system and thereafter receive generated security information needed to access the system. A profile is created for each registered organization, through which the organizations can access the modules of the system. The profile displays the modules of the system, individuals connected with the organization, suborganizations, and the groups the organization has created and joined. The profile typically includes organization details including name, field(s) of interest, resources, qualifications and the like. The initiation module 105 is configured to be accessed by a registered organization to initiate a discussion, a program, or a project. Other organizations can be invited to participate in discussions and share their views with the initiating organization.

The intelligent query module 107 is configured to retrieve information associated with an organization's profile, as well as organization-related documents, research results and publications, development updates, medicine in use in clinical trials and the like. The intelligent query module 107 applies an automated learning algorithm to generate and index the information from a variety of sources that are stored in the database as well as other sources that are interfaced to the system, such as the internet, proprietary and public databases, and storage drives (e.g., google drive, one-drive) given adequate permissions. The automated learning algorithm is configured to utilize artificial intelligence methodologies, self-learning methodologies and other intelligent methods to retrieve information relevant to a search query. The intelligent query module 107 in combination with an automated learning algorithm automatically updates, and indexes information which can be displayed on a screen rendered by the dynamic display means 103.

In accordance with one embodiment of the invention an organization registered with the system can interact seamlessly with other registered organizations. Each organization may connect with another by sending or receiving an join group request to be added to a group profile members list.

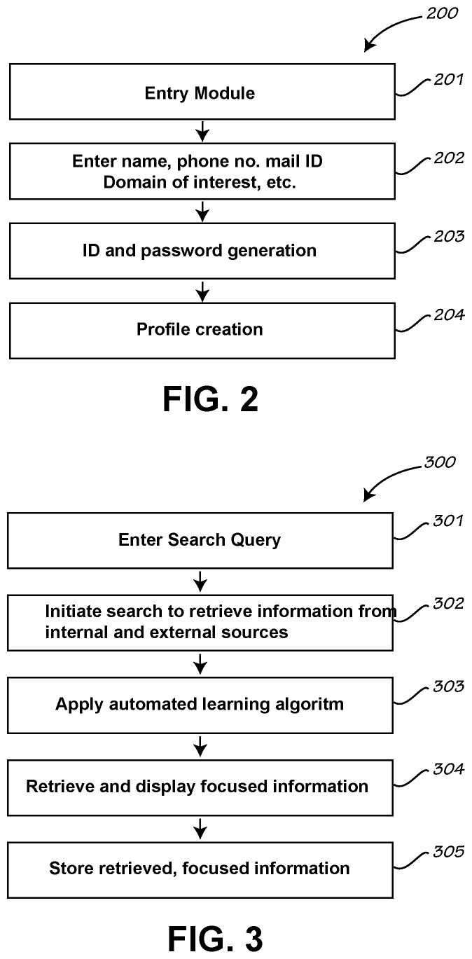

As illustrated in FIG. 2, the organizations of the system register 200 with the system by entering appropriate identifying credentials. At step 201, the organization accesses the entry module of the system. Accessing the entry module initiates communication between the organization and the system and an entry screen is rendered and displayed on an organization device. A new organization registers with the system by accessing the entry screen generated by the entry module and rendered by the dynamic display means in step 202. The rendered entry screen, which can be shown on a display of an organization's desktop computer, laptop computer, tablet, mobile phone, smart TV or the like, prompts the organization to enter a name, email address, physical address, phone number and other authenticating information on the graphical user interface. In some aspects of the invention, the authentication information includes a code, phrase, or temporary password that has been sent by the system to the organization. In certain aspects of the invention, the prompt is a word(s) or sentence(s) instructing the organization to fill in blanks. In other aspects, the entry model generates an audible voice message of the instruction that is transmitted to the organization's device. The organization then types or dictates the authentication information on his or her device, which communicates the information to the system. The information is compared to the authentication information stored in a storage location for the organization.

If a match is found, the entry module generates a user ID and password for the organization (step 203), which may follow a pattern of characters, may include random characters, or may be a combination thereof. The user ID and password are stored in an indexed storage location of the system and transmitted to the organization as described above. To login subsequently for access to the system, the organization enters the generated user ID and password (as described above), and the entry module compares the entered user ID and password to the saved user ID and password. If a match is detected by the entry module, the entry module makes additional screens of the entry module and additional modules visible and available to the organization.

At step 204, a profile creation interface (saved in storage) is retrieved and made available by the completion of registration or entry of user ID and password, and is displayed on the organization's device. The organization is then prompted to enter additional information to create an organization profile, which can include a plurality of information related to the organization. An unlimited kind and amount of information can be entered to fully characterize the organization, its interests, capabilities, expertise, previous accomplishments, current endeavors and the like. Entry of an organizations' profile information can be accomplished manually, automatically or by a combination thereof. Manual entry involves retrieval of information from offline sources, which may include confidential and proprietary information, whereas automated entry involves retrieval of information from one or more online sources such as search engines (e.g. google), websites (e.g. the organization's website(s)) and databases (e.g. a National Center for Biotechnology Information (NCBI) or National Library of Medicine (NLM) databases such as PubMed or OMIM, a Thomson-Reuters database or the like), and confirmation of entry by the organization. In some instances, the entry module may retrieve different versions of the same information, such as an older version and a newer version. In certain aspects, the entry module compares the versions and either combines non-overlapping information or selects the most recent or most comprehensive version of the same information.

The profile affords display and viewing of information in various visual representations (e.g., Map Views, Bubble or Cluster View(s), and List or Group Views), sharing information, interacting with other profile organizations throughout the world, forming groups of organizations, suborganizations and individuals, and utilizing the additional functionalities included in the system and interfaced thereto. Advantageously, the various views provide customizable and intuitive visual representations that an organization can immediately appreciate via visual cues of color, shape, size, distance and the like. Profile information entered manually or automatically is saved by the entry module to a profile storage location indexed to the organization and category of information. In certain embodiments of the invention, the category of profile information is selected from one or more lists stored in memory and displayed by the entry module. For example, the type of organization (described above) can be a presented as a list or outline, which may be displayed e.g. as a drop-down menu, from which a type can be selected. The entry module also provides the option to add new types (or other categories of information) which are then saved to a storage location associated with the entry module and incorporated into the entry module. In this way, the entry module (as well as other modules of the system) can “learn” from user input. In other aspects, the profile includes information that cannot be categorized or is unique to an organization, which can also be saved to an organization's profile.

It will be appreciated that variants of the above-disclosed registration procedure and other features and functions or alternatives thereof that are associated with the procedure, may be advantageously combined into various modules, applications and methods directed to and to be used by a wide variety of organizations and/or individuals. Procedural steps disclosed are exemplary only, and the order of procedural steps can be altered. Additional procedural steps may be added as needed to customize and optimize the efficacy of the collaboration research system.

According to an embodiment of the invention, an organization related to healthcare such as one operating in the oncology domain, can interact using the unifying platform provided by the system and methods described herein. Interaction between organizations (including sub-organizations and individuals as detailed above), is desirable and may be necessary to promote research objectives, maintain currency in latest developments and build contact sources for expertise, funding and the like.

In accordance with an exemplary embodiment of the invention, registered organizations can access the initiation module of the invention to interact, have discussions and participate in other group activities. The initiation module, when executed by an organization, provides a graphical interface for accessing messaging, message board, chat, conference, including audio and video conferencing, tools that are interfaced with the platform.

In accordance with another embodiment of the invention, organizations can access the sharing module of the invention to share documents, files (including graphic, audio, video and multimedia files) and the like to be viewed by other organizations. The organizations can select the sharing module and exchange information with another organization. The ability to share information with other organizations facilitates the access to important data which would otherwise require organizations to manually intervene with disparate sources/applications to access that data. For example, a video file may require a specific player, such as Windows Media Player or Adobe Flash Player, that may not be available on the recipient's phone or computer. According to certain aspects of the invention, the relevant player is associated with the data and made available to the recipient on the unifying platform of the system of the invention. This feature facilitates information exchange between disparate organizations that can be made available on the single platform of the invention.

FIG. 3 is a flowchart illustrating the steps 300 for retrieving information using the intelligent query module of the invention. At step 301, the registered organization enters a search query of the information required. The intelligent query module facilitates retrieval and display of information relevant to the query from a variety of sources, including as information from publicly available online databases. by executing the steps of transmitting the query to an internet search engine. and retrieving the results of the search. The intelligent query module organizes retrieved information and stores it in a unique storage location indexed to the both the search and the user. The intelligent query module can also access information stored within the storage module and linked databases of the system. Information that may be searched and retrieved includes, but is not limited to knowledge pertinent to a specific organization; location; employee identification; products and services provided; research and technology interests; partners; development plans; and the like. At step 302, upon entry of a the search query, a search is initiated by the intelligent query module, which compares the query to information stored in the storage module of the system, databases linked thereto, and non-system sources of information (e.g., information on the internet), by matching the entered search query with the stored internal and external information, and retrieving matching information from internal and external sources. At step 303, the intelligent query module applies an automated learning algorithm to retrieve the target or relevant information based on the search query. Initially, the automated learning algorithm retrieves information matching the search input query. The user (i.e., organization) then responds to a sample of the retrieved information by confirming relevancy or indicating that the information is non-relevant. At step 304 the automated learning algorithm then identifies features common to relevant information, and sorts the information on this basis, thereby excluding non-relevant information, and displaying only the relevant information for review. Step 304 can be repeated as necessary to further focus the results. Finally, the retrieved information is stored in a storage location indexed by the user-inputted query and additional parameters determined by the focusing process Step 305. In certain aspects, the additional parameters determined by the focusing process may be saved to a storage location indexed to the user, and applied to future searches executed by the intelligent query module for that user.

In certain exemplary embodiments of the invention, an organization or an individual is able to retrieve information utilizing the intelligent query module using a prescribed user- or organization-specific process that incorporates previously determined parameters. For example. a user may repeatedly reject as non-relevant certain initial search results, e.g. that are from a foreign country or relate to a particular patient population. The user may not be aware of the correlations amongst rejected results, but the computer learning algorithm may detect and correlate those distinctions to the user's preferences. New and/or improved search queries and procedures may be developed and integrated as new processes; and/or developed as modules to be integrated into existing process, supporting one or more modules described above; or may standalone. Moreover, the procedures may be developed and/or integrated in a more secured and customized manner pertinent to a particular organization and/or an individual.

In accordance with yet another embodiment of the invention, the intelligent query module stores the retrieved information in a storage module and further updates the storage module. The intelligent query module, by applying an automated learning algorithm, may select and classify the retrieved information. The selected information may then be reviewed and classified as relevant or as non-relevant by the organization, and this additional classification used to update the classifier, thereby further improving the effectiveness of the intelligent query module along with the automated learning algorithm to retrieve focused results.

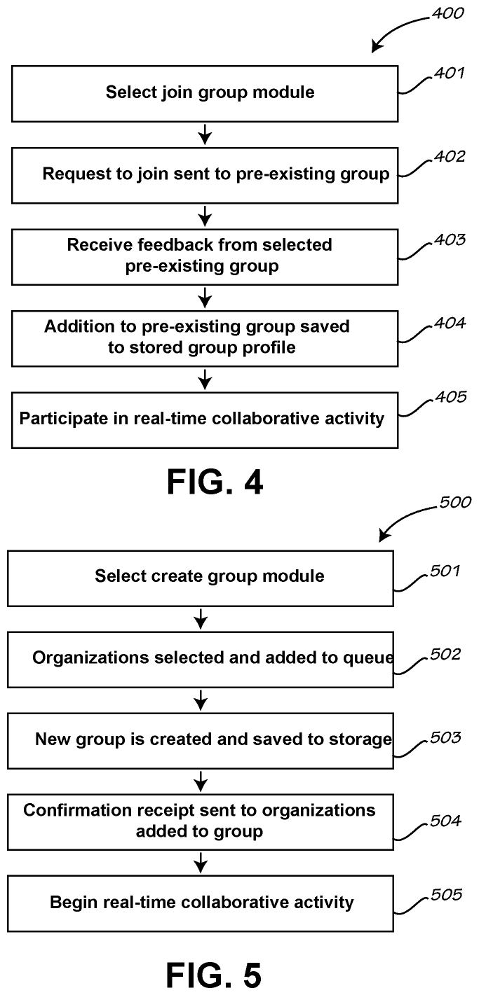

FIG. 4 is a flowchart illustrating a procedure 400 for an organization to join a pre-existing group using the join group module of the invention, which is included within the group activity module. At step 401, the join group module is selected by a registered organization and an existing group of collaborative interest is selected by the registered organization from the collection of existing groups saved to and listed by the module. At step 402, the join group module transmits a request to the existing group selected by the registered organization. At step 403, feedback is received by the module, and transmitted to the registered organization. If the existing group accepts the new (registered) organization, the change (addition) is saved to the stored profile of the existing group (step 404). At step 405, the organization added to the existing group is permitted to participate in real-time collaborative activity within the existing group.

For instance, in the above example, an organization or an individual is able to join a pre-existing group, using a prescribed exemplary process; whereas new and/or improved procedures may be developed and integrated as new processes and/or developed as modules to be integrated into existing process supporting one or more modules described above or may standalone. Moreover, the procedures may be developed and/or integrated in a more secured and customized manner pertinent to a particular organization and/or an individual.

FIG. 5 is a flowchart illustrating a process 500 for creating a new group of at least two different organizations using the create group module that is present within the group activity module. At step 501, a registered organization wishing to create a new group (the initiating organization) selects the create group module. At step 502, the module lists the collection of organizations within the system, from which the initiating organization can select to form a new group. One or more organization is selected by the initiating organization from those listed and the profiles of those groups are added to a queue by the create group module. At step 503. a new group is created that includes the selected one or more organizations and the initiating organization, and a group profile identifying all organizations in the group is stored and indexed to the profiles of each organization of the group. Optionally, the initiating organization can create a name for the group, which is also stored to the group profile. At step 504, a confirmation receipt is sent by the module to the organizations comprising the new group informing the organizations of group creation. Thereafter, all members of the group can, at step 505, initiate and participate in real-time collaborative inter-organizational activity between and amongst the organizations within the created group.

Methods for creating groups across organizations are key features of the system because collaboration is vital to successful research and development. Methods of group creation can include other process or steps and can be customized to achieve the purpose of group creation across organizations or to make the system more effective without deviating from the scope of the invention. For example, an organization can indicate types of organizations it may be interested in and which organization types it is not interested in for the purpose of collaborating. Those preferences are stored in the organization's profile, and will appear when the organization is contemplated for inclusion in a new group. In one aspect of the invention, the organization can decline certain group types even before it is invited to join a particular group. According to such aspects, the create group module can be instructed to remove an organization's name from lists of organizations available for inclusion when the group is of a certain type disfavored by the organization.

The methods of group creation disclosed herein are illustrative and exemplary of situations where group creation facilitates interaction between organizations. If at the time a group is formed or any time thereafter, an organization no longer wishes to participate in created group, a removal instruction can be selected by that organization within the group activity module. The change will be saved to the stored group profile and notification of the removal sent to the remaining members of the group.

In accordance with another embodiment of the invention, the collaboration research platform provides group-specific organization profiles that may utilize separate authentication in lieu or in addition to the organization's authentication and profile, to limit accessibility of certain organizational information to one group but not to another group that the organization is a member of. In another embodiment. group-specific organization profiles permit the group to focus more narrowly on a particular project or program which may have different goals, resources and needs than the broader organization or another group. The various alternatives for authentication, sharing and profile development further allow information to be viewed, discussed and shared among the relevant members of a group or between groups. Different tools can be utilized within the platform to enhance or restrict collaboration between the groups.

Among the real-time collaborative activities that groups can participate in are document and data sharing activities that interface with the various visually compelling data views to provide insight and highlight concurrent objectives and synergies of organizations within group. Also available are project management tools that facilitate milestone development, identify responsibilities, track timelines and prepare budgets for organizations with a group working together toward mutual goals of a collaborative project.

The collaboration platform additionally allows for integration of the third-party applications. For example, third-party applications may include those that interface to social media, social interaction and networking systems, electronic commerce, medical software applications, advanced product trends, and surgical method trends. The social interaction technologies contemplated for integration include Facebook, Twitter, chat, text messaging, E-mail technologies, voice mail, direct voice communication, video conferencing and the like, and may involve multiple modes such as phone, personal computer, tablet device, and television. The social interaction may persist between organizations and also between products and organizations to further solve problems or propose improvements collectively. Therefore, the collaboration platforms can be integrated with various third-party applications and thereby facilitate effective collaboration between the groups.

The recited modules of the invention are not intended to be limiting. Those described herein in detail represent a minimum set of instructions to supply the basic functions of the system. Additional modules can be added, and the recited modules expanded to provide additional functionality, connectivity and to customize the system for specific uses. In addition, the skilled artisan will appreciate that application specific tool sets, widgets, add-ins and the like can be added or interfaced to the platform under certain circumstances of use.

The invention facilitates organizations to interact, initiate projects and monitor their progress in a seamless manner. The invention provides a means in the unifying platform for retrieval of information from various online sources by the organizations. It should be understood that the collaborative research platform may be employed in any type of operating system. The present invention may be implemented by any type of software code using any language and can run on any type of computer hardware or networked computer hardware, including virtual machines. The computer hardware, virtual or physical, may include a processor, a program memory, and a data storage. The computer hardware may be networked, wired and wireless, to other computer hardware and accessible via other electronic devices, such as smart phones, PDAs and the like.

The inventive collaboration research system for organizations disclosed above gives an overview of different modules of the system needed for organizations to collaborate effectively for better research goals. Further for understanding the scope and inventive step of the collaborative research'system it is explained exclusively considering organizations but the same system can be extended or customized for individuals of organizations or for research individuals interested in specific research the system is intended for. The customization to the system may include steps disclosed in the invention and may further include steps not disclosed in order to attain proper collaborative research system and platform for individuals.

The collaboration research system of the invention therefore engages different stakeholders such as organizations, individuals and groups to organize different research sectors or other sections in a collaborative manner related to a particular domain. The platform also supports organizations in implementing the research sections and collaborative endeavors in a sequential way without departing from individual or organizational interests, thus ultimately resulting in the overall community growth.

The inventive collaboration research system further also includes aspects of privacy and personal protection. Each module, profile, intelligent query and the like includes customizable permissions that allow the user and organization to determine which information is to be shared and with whom. Particularly sensitive, and high-value confidential information can be protected with multiple layers of authentication, such as token-based random number generation, biometrics, PKI certificates, and the like.

It will readily be apparent that numerous modifications and alterations can be made to the processes described in the foregoing examples without departing from the principles underlying the invention, and all such modifications and alterations are intended to be embraced by this application.

1-38. (canceled)

39. A system 100 for research collaboration comprising:
a) a non-transitory computer-readable storage medium 101 storing a plurality of modules, each module comprising instructions that when executed on a processor 10, which is optionally a cloud-based processor, cause the processor 10 to perform a series of steps, wherein the plurality of modules comprises: a storage module 102, an entry module 104, an initiation module 105, a sharing module 106, an intelligent query module 107 and a group activity module 108;
b) a dynamic display means 103, and
c) a database 102a.

40. The system 100 of claim 39, wherein: the dynamic display means 103 is configured to render a graphical user interface on a display device, optionally selected from a computer monitor, a tablet, a smart television and a mobile phone

41. The system 100 of claim 40, wherein the graphical user interface is a map view, a bubble view, a cluster view, a group view, a list view or tabular view.

42. The system 100 of claim 39, wherein:
a) the storage module 102 is configured to receive, index, and store information on the non-transitory computer-readable storage medium 101 and to retrieve indexed and stored information from the non-transitory computer-readable storage medium; and/or
b) the information is stored in the database 102a, which is optionally a relational database optionally linked to remote data storage on a cloud-based.

43. The system 100 of claim 39, wherein the entry module 104 is configured to receive categorical information about organizations, sub-organizations and individuals, and to authenticate and register the organizations, sub-organizations and individuals, thereby permitting access to all or a portion of the modules of the system 100.

44. The system 100 of claim 39, wherein the initiation module 105 is configured to initiate communication, projects, and programs between organizations, sub-organizations and individuals.

45. The system 100 of claim 39, wherein the sharing module 106 is configured to enable organizations, sub-organizations and individuals to exchange information.

46. The system 100 of claim 39, wherein the intelligent query module 107 is configured to perform query-based searches and locate, store and retrieve information relevant to a query.

47. The system 100 of claim 39, wherein the intelligent query module 107 perceives, learns, remembers and adapts queries based on query history and user input or uses artificial intelligence.

48. The system 100 of claim 39, wherein the group activity module 108 is configured to allow organizations, sub-organizations and individuals to create new groups and to join existing groups and optionally comprises a create group module and a join group module.

49. The system 100 of claim 48, wherein:
a) the organizations, sub-organizations and individuals within a group can initiate and participate in real-time collaborative activity among other organizations, sub-organizations and individuals in the group; and
b) the real-time collaborative activity is optionally selected from the group consisting of: document sharing, data sharing, entering into collaborative projects, managing collaborative projects, messaging via message boards, direct messaging, audio conferencing and video conferencing.

50. The system 100 of claim 48, wherein:
a) the organizations are selected from the group consisting of colleges; universities; medical schools; research institutes; foundations; human and veterinary hospitals; human and veterinary clinics; pharmaceutical companies; and biotechnology companies; medical device research, development and manufacturing companies; diagnostic laboratories; reference laboratories; contract research organizations; government institutes; government laboratories; government agencies; venture capitalist firms; investment banks; philanthropic groups; philanthropic societies; disease-focused patient groups; and professional associations;
b) the sub-organizations are selected from the group consisting of: a university department; a hospital department; a company department; a division; a subsidiary; a regional campus; a regional office; a regional building; regional satellite; an individual research laboratory; and a core shared facility operation; and/or
c) the individuals are selected from the group consisting of professionals; physicians; surgeons; specialists; scientists; veterinarians; officers; directors; managers; stakeholders; administrators; consultants; attorneys; content contributors; curators; librarians; moderators; involuntary supporters; voluntary supporters; researchers; investigators; technicians; professors; lecturers; undergraduate students; graduate students; medical students; inventors; project managers; grant writers; and technical writers.

51. A computer-implemented method for collaboration between organizations, comprising the steps of:
a) registering two or more organizations for access to the system 100;
b) creating a profile for each organization, wherein the profile includes information selected from the group consisting of: the name of the organization, the type of organization, the domain or domains of the organization, the address of the organization, the size of the organization, the qualifications, expertise and proficiencies of the organization, the research interests of the organization, the resources of the organization, and the needs of the organization;
c) dynamically displaying all or a portion of each organization's profile in a map view, a bubble view, a cluster view, a group view, a list view or tabular view; and
d) creating at least one group of the two or more organizations, wherein the organizations in the group initiate at least one real-time collaborative activity.

52. The computer-implemented method of claim 51, wherein the real-time collaborative activity is selected from the group consisting of: document sharing, data sharing, entering into collaborative projects, managing collaborative projects, messaging via message boards, direct messaging, audio conferencing and video conferencing.

53. The computer-implemented method of claim 51, wherein the organizations comprise sub-organizations and individuals, wherein:
a) the organizations are selected from the group consisting of colleges; universities; medical schools; research institutes; foundations; human and veterinary hospitals; human and veterinary clinics; pharmaceutical companies; and biotechnology companies; medical device research, development and manufacturing companies; diagnostic laboratories; reference laboratories; contract research organizations; government institutes; government laboratories; government agencies; venture capitalist firms; investment banks; philanthropic groups; philanthropic societies; disease-focused patient groups; and professional associations;
b) the sub-organizations are selected from the group consisting of: a university department; a hospital department; a company department; a division; a subsidiary; a regional campus; a regional office; a regional building; regional satellite; an individual research laboratory; and a core shared facility operation; and/or
c) the individuals are selected from the group consisting of professionals; physicians; surgeons; specialists; scientists; veterinarians; officers; directors; managers; stakeholders; administrators; consultants; attorneys; content contributors; curators; librarians; moderators; involuntary supporters; voluntary supporters; researchers; investigators; technicians; professors; lecturers; undergraduate students; graduate students; medical students; inventors; project managers; grant writers; and technical writers.

54. A system 100 for research collaboration comprising a computer-readable medium comprising instruction that when executed on a processor, performs the steps of
a) registering at least one organization on the system 100;
b) storing information about the registered organization in a profile on a non-transitory computer-readable storage medium; and
c) rendering and dynamically displaying all or part of the information in the profile.

55. The system 100 of claim 54, wherein displaying all or part of the information in the profile comprises displaying a map view, a bubble view, a cluster view, a group view, a list view or a tabular view of all or part of the information in the profile.

56. The system 100 of claim 54, wherein the organizations comprise sub-organizations and individuals, wherein:
a) the organizations are selected from the group consisting of colleges; universities; medical schools; research institutes; foundations; human and veterinary hospitals; human and veterinary clinics; pharmaceutical companies; and biotechnology companies; medical device research, development and manufacturing companies; diagnostic laboratories; reference laboratories; contract research organizations; government institutes; government laboratories; government agencies; venture capitalist firms; investment banks; philanthropic groups; philanthropic societies; disease-focused patient groups; and professional associations; and/or
b) the sub-organizations are selected from the from consisting of: a university department; a hospital department; a company department; a division; a subsidiary; a regional campus; a regional office; a regional building; regional satellite; an individual research laboratory; and a core shared facility operation; and/or
c) the individuals are selected from the group consisting of: professionals; physicians; surgeons; specialists; scientists; veterinarians; officers; directors; managers; stakeholders; administrators; consultants; attorneys; content contributors; curators; librarians; moderators; involuntary supporters; voluntary supporters; researchers; investigators; technicians; professors; lecturers; undergraduate students; graduate students; medical students; inventors; project managers; grant writers; and technical writers.

57. The system 100 of claim 54, wherein registering at least one organization on the system 100 comprises the steps of:
a) selecting an entry module 104 comprising instructions that when executed on a processor 10, cause the processor 10 to:
(i) prompting the organization to enter authenticating information selected from the group consisting of name, address, mail ID, a security code, and domain of interest;
(ii) comparing the authenticating information to authentic information for the organization;
(iii) authenticating the organization when the information of i) and ii) match; and
(iv) generating a user ID and password;
b) entering additional profile information of the organization; and
c) storing the profile information, user ID and password to an indexed location on a non-transitory computer-readable storage medium.

58. The system of claim 54, further comprising the organization
a) conducting a search comprising the steps of:
(i) entering a search query;
(ii) initiating a search to retrieve information related to the search query;
(iii) applying an automated learning algorithm to retrieved information to generate a focused search;
(iv) retrieving and displaying the results of the focused search; and
(v) storing the focused search results to an indexed location on a non-transitory computer-readable storage medium;
b) creating a group comprising the steps of:
(i) selecting at organizations to form a group;
(ii) adding the selected organizations to a queue;
(iii) saving a profile of the group of selected to organizations to an indexed location on a non-transitory computer-readable storage medium; and
(iv) beginning a real-time collaborative activity among the organizations in the group, wherein the activity is optionally selected from sharing documents, sharing data, entering into collaborative projects, managing collaborative projects, messaging via message boards, messaging directly, conferencing via audio and conferencing via video; and/or
c) joining an existing group comprising the steps of:
(i) requesting to join the existing group;
(ii) receiving an acceptance response from the existing group;
(iii) adding the organization to the existing group;
(iv) saving a modified profile of the group that includes the added organization to an indexed location on a non-transitory computer-readable storage medium; and
(v) initiating a real-time collaborative activity among the organizations in the group.
